(19) United States
(12) Patent Application Publication
(10) Pub. No.: US 2008/0234351 A1
(43) Pub. Date: Sep. 25, 2008

(54) Combination Methods for Preserving Visual Acuity

(76) Inventors: Matthew John Sheetz, Indianapolis, IN (US); Louis Vignati, Indianapolis, IN (US); Xin Zhi, Indianapolis, IN (US)

Correspondence Address: ELI LILLY & COMPANY, PATENT DIVISION, P.O. BOX 6288, INDIANAPOLIS, IN, 46206-6288, US

(21) Appl. No.: 12/064,883

(22) PCT Filed: Sep. 18, 2006
(86) PCT No.: PCT/US2006/036505

§ 371 Date: Feb. 26, 2008

Related U.S. Application Data
(60) Provisional application No. 60/721,004, filed on Sep. 27, 2005.

(51) Int. Cl. A61K 31/407; A61P 3/10; A61P 27/02

(52) U.S. Cl. 514/410

(57) ABSTRACT

The present invention relates to a method for preserving the vision of a patient suffering from diabetic retinopathy which comprises administering to said patient an effective amount of ruboxistaurin or a pharmaceutical salt thereof wherein said administration is in conjunction with focal or grid laser photocoagulation therapy.

### BACKGROUND OF THE INVENTION

Diabetic retinopathy (DR) is the leading cause of new cases of blindness in the western world. The most common cause of visual impairment in DR is diabetic macular edema (DME). DME is the accumulation of extracellular fluid in the retinal tissues of the macula. DME is classified by the Early Treatment Diabetic Retinopathy Study (ETDRS) criteria, depending on the prognostic value, into two groups: non-clinically significant macular edema (NCSME) and clinically significant macular edema (CSME).

If untreated, 30% of patients with CSME will develop significant loss of central vision within 3 years (ETDRS Report Number 2. Opthalmology 94(7): 761-774, 1987a). When CSME becomes vision-threatening, it is usually treated with focal or grid laser photocoagulation. Though laser therapy can help to reduce severe vision loss, it is generally not effective in restoring visual acuity (Aiello L P, Surv. Opthalmol. 47(suppl2): S263-S269, 2002). Laser therapy can have many significant side effects including decreased color perception (Birch and Hamilton, Trans. Opthalmologic Society UK, 101(1): 93-99, 1981), loss of peripheral vision and worsening of visual acuity (Frank, Arch. Opthalmoll., 93: 591-598, 1975). These side effects, coupled with the facts that the efficacy rate (prevention of subsequent vision loss) for focal/grid photocoagulation is approximately 50% (ETDRS Report Number 4, Int. Opthalmol. Clin., 27(4): 265-272, 1987b), the lack of efficacy from any treatment in restoring vision once lost and the lack of any approved pharmaceutical treatment for DME, necessitate further research for additional therapy for vision loss associated with DME.

### BRIEF SUMMARY OF THE INVENTION

The present invention relates to a method for preserving the vision of a patient suffering from diabetic retinopathy which comprises administering to said patient an effective amount of ruboxistaurin or a pharmaceutical salt thereof wherein said administration is in conjunction with focal or grid laser photocoagulation therapy.

### DETAILED DESCRIPTION OF THE INVENTION

The present invention further relates to a method for preserving the vision of an adult patient suffering from diabetic retinopathy and macular edema which comprises administering to said patient an effective amount of ruboxistaurin or a pharmaceutical salt thereof wherein said administration is in conjunction with focal or grid laser photocoagulation therapy.

The present invention further relates to a method for preserving the vision of an adult patient suffering from moderate to severe non-proliferative diabetic retinopathy (NPDR) and CSME which comprises administering to said patient an effective amount of ruboxistaurin, or a pharmaceutical salt thereof wherein said administration is in conjunction with focal or grid laser photocoagulation therapy.

Ruboxistaurin is also known as: (S)-9-((dimethylamino)methyl)-6,7,10,11-tetrahydro-9H,18H-5,21:12,17-dimethenodibenzo(e,k)pyrrolo(3,4-h)(1,4,13)oxadiazacyclohexadecine-18,20(19H)-dione. The mesylate monohydrate of ruboxistaurin is currently in Phase III clinical trials for various microvascular complications of diabetes and is structurally depicted as:

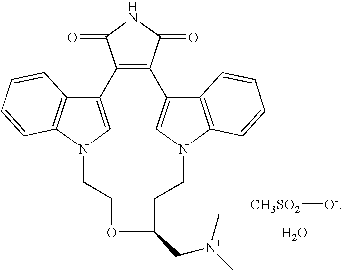

Ruboxistaurin, its pharmaceutically acceptable salts and related compounds are described in Heath, Jr., et al., U.S. Pat. No. 5,552,396. The mesylate salts of ruboxistaurin are specifically described and claimed in U.S. Pat. No. 5,710,145. The synthesis of ruboxistaurin, its salts and related compounds as well as a disclosure that said compounds are useful in the treatment of conditions associated with diabetes mellitus and its complications as well as ischemia, inflammation, central nervous system disorders, cardiovascular disease, dermatological disease, Alzheimer's disease and cancer may also be found in U.S. Pat. Nos. 5,552,396 and 5,710,145. U.S. Pat. Nos. 5,552,396 and 5,710,145 are hereby incorporated by reference in their entirety as if fully set forth herein.

The term “pharmaceutical” when used herein as an adjective means substantially non-deleterious. In the context of a pharmaceutical salt, it should be recognized that the particular counterion forming a part of any salt of this invention is usually not of a critical nature, so long as the salt as a whole is pharmacologically acceptable and as long as the counterion does not contribute undesired qualities to the salt as a whole.

As used herein, the term “preserving” is defined to include its generally accepted meaning and also includes reversing the progression of vision loss. Preservation of vision in the context of this invention means that the visual acuity of patients undergoing therapy according to the present invention will be improved, maintained, or will regress at a slower rate than in patients not undergoing therapy. Visual acuity is determined using the “Visual Acuity Grading Scale” discussed below.

As used herein, the term “effective amount” means an amount of ruboxistaurin that is capable of preserving the vision of a patient suffering from diabetic retinopathy as herein described. The specific dose of a compound administered according to this invention will, of course, be determined by the particular circumstances surrounding the case including, for example, the route of administration and the state of being of the patient. A preferred dose range for ruboxistaurin mesylate monohydrate is from 32 mg to about 128 mg, administered once per day orally. Most preferably, the dose of ruboxistaurin mesylate monohydrate will be 32 mg/day.

As used herein, the phrase “in conjunction with” means that the patient undergoing therapy according to the present invention will at some time in the past, present or future, relative to the commencement of therapy with ruboxistaurin (or salt thereof), have focal or grid laser photocoagulation therapy treatment of their diabetic retinopathy. Preferably, the patient receives laser therapy within one to three months, prior to, or after, commencement of ruboxistaurin therapy. More preferably, the patient receives laser therapy within one month prior to, or after, commencement of ruboxistaurin therapy with one to four weeks prior to being most preferred.

A preferred patient population contemplated for therapy by the present invention are type 1 and/or type 2 diabetic humans suffering from diabetic retinopathy whose retinopathy falls within the “moderately severe” to “very severe” categories of non-proliferative diabetic retinopathy. These categories of patients are further described in the “Final Retinopathy Severity Scale” below.

### EXAMPLE 1

### Clinical Study MBCM(b)

MBCM(b) is a Phase 3, multicenter, parallel, randomized, double-masked, placebo-controlled study. Language from the MBCM(b) protocol is included below.

Patients with type 1 or type 2 diabetes mellitus and an Early Treatment Diabetic Retinopathy Study (ETDRS) retinopathy level ≧47A and ≦53E in at least one eye will be eligible to participate in the study. Visual acuity (best-corrected) will be assessed using the ETDRS visual acuity protocol. Retinopathy will be assessed using ETDRS 7 standard field 30 degree color stereoscopic fundus photography. The photographs will be independently assessed by the University of Wisconsin Fundus Photograph Reading Center.

Seventy sites have enrolled patients over a 15-month period. Of the 685 patients enrolled, it is expected that approximately 580 patients will complete the study. Both eyes of all patients will be followed throughout the study. Patients will remain on study medication and maintain regularly scheduled study follow-up visits for the duration of the trial even if they receive laser photocoagulation for diabetic macular edema (DME) or proliferative diabetic retinopathy (PDR).

This study will consist of three phases: a screening and randomization phase which will last up to 6 weeks, a treatment phase which will last 36 months, and an extended treatment phase which will last 0 to 6 months depending on when the patient was enrolled.

Study drug is to be administered orally with a meal once a day for the duration of the study. Patients will take one tablet of study drug per dose once daily. The meal with which study drug is taken is typically the largest meal of the day for the patient. Thus, the daily time of ingesting study drug may vary from patient to patient depending upon their meal pattern. Ruboxistaurin mesylate monohydrate will be administered as tablets containing 32 mg of said mesylate monohydrate.

### Inclusion Criteria

Patients may be included in the study only if they meet all of the following general criteria and have at least one eye that meets all of the eye-specific criteria:
[1] Type 1 or type 2 diabetes mellitus as defined by American Diabetes Association (ADA) or World Health Organization (WHO) Classification of Diabetes criteria.
[2] ETDRS retinopathy level ≧47A and ≦53E without previous panretinal photocoagulation as determined using ETDRS 7 standard field 30-degree color stereoscopic fundus photography.
[3] 18 years of age or older at Visit 1 (Week-6).
[4] Hemoglobin Alc ≦13.0% during the screening and randomization phase.
[5] Without language barrier, cooperative, and agree to return for all follow-up visits and to give informed consent before entering the screening and randomization period, after being informed of the medications and procedures to be used in the study.
[6] Free of severe or chronically disabling conditions other than diabetes mellitus, moderately severe to very severe NPDR, and DME.
[7] Best-corrected visual acuity score of 45 or more letters as measured using the ETDRS visual acuity protocol (ETDRS Study report number 1, Arch. Opthalmol., 103:1796-1806, 1985) in an eye that has level ≧47A and ≦53E retinopathy.

### Exclusion Criteria

Patients will be excluded from the study if they meet any of the general criteria listed below or if the only otherwise eligible eye meets any of the eye-specific criteria listed below:
[1] History of panretinal photocoagulation for DR.
[2] The presence of occludable chamber angle or of glaucoma in the opinion of the investigator. Ocular hypertension in the absence of a glaucomatous visual field defect is not an exclusion criterion.
[3] A history of conditions which might affect the progression of DR in the opinion of the investigator, including intraocular surgery (including cataract extraction within six months prior to Visit 1 or neodinium yttrium aluminium garnet (Nd:YAG laser) capsulotomy within 6 months prior to Visit 1), significant chorioretinal scars, optic atrophy, retinal degeneration, retinal vein occlusion, retinal artery occlusion, rubeosis iridis, pathologic myopia, etc.
[4] Current vitreous or preretinal hemorrhage.
[5] Inability to obtain adequate fundus photography (as assessed by the fundus photograph reading center) due to media opacities or compliance, for example.
[6] Current history of unstable angina (as defined by the Braunwald system).
[7] Sitting systolic blood pressure ≧190 mm Hg or sitting diastolic blood pressure ≧105 mm Hg as determined by the mean of three separate measurements at Visit 1.
[8] QTc prolongation of >500 msec or a second degree or higher heart block on ECG obtained during the entry phase of this study.
[9] Abdominal, thoracic, vascular, or cranial surgery that is determined to be of major significance by the investigator within 3 months prior to Visit 1.
[10] Currently suspected carcinoma or treatment for cancer within 6 months prior to Visit 1 or anticipated treatment for cancer during the course of the study, with the exception of excised superficial lesions such as basal cell carcinoma and squamous cell carcinoma of the skin.
[11] Poor medical or psychiatric risk for treatment with an investigational new drug, in the opinion of the investigator.
[12] Treatment with a drug within the last 30 days that has not received regulatory approval at the time of study entry.
[13] Pregnant or intent to become pregnant during the time of the study.
[14] A sexually active woman of childbearing age not actively practicing birth control by using a medically approved device or therapy (that is, intrauterine device, oral contraceptive, implant, Depo-Provera, or barrier device.)
[15] A woman who is breast feeding.
[16] Any other findings that, in the opinion of the Investigator, would preclude the patient's participation in the study.

### Disease Diagnostic Criteria

Patients with type 1 diabetes will be defined as those patients with an onset of diabetes before age 30 and who have been treated with the continuous use of insulin since their diagnosis. Patients with type 2 diabetes will be defined as all patients with diabetes not meeting the definition for type 1 diabetes.

Moderately severe to very severe NPDR is defined as Early Treatment Diabetic Retinopathy Study (ETDRS) level of ≧47A and ≦53E as determined using ETDRS 7 standard field 30 degree color stereoscopic fundus photography and the modified Airlie House classification system.

Definite proliferative diabetic retinopathy (PDR) is defined as ETDRS level of ≧65 as determined using ETDRS 7 standard field 30 degree color stereoscopic fundus photography and the modified Airlie House classification system.

Panretinal (scatter) photocoagulation is defined as the application of photocoagulation for the purpose of treating very severe nonproliferative or PDR as reported by the investigator.

### The Need for Laser Surgery

The ultimate decision as to whether a patient receives laser photocoagulation resides with the study investigator and the patient. However, it is expected that the study investigator will only rarely initiate panretinal photocoagulation prior to the development of ETDRS level 65 or higher proliferative retinopathy. The investigator is not required to initiate laser panretinal photocoagulation when this level of PDR develops.

When the investigator decides that initiation of panretinal photocoagulation should be considered, 7 standard field photographs of both eyes should be obtained and sent promptly to the fundus photograph reading center, with notification that these are pretreatment photographs. If this decision is made at a regularly scheduled visit, the visit photographs will serve this purpose. If this decision is made at an unscheduled visit, photographs are to be taken in conjunction with that visit.

Whenever clinically appropriate, the investigator should request fundus photograph reading center evaluation of the pretreatment photos and wait for feedback prior to initiating treatment.

If treatment is initiated, an additional set of 7 standard field photographs should be taken prior to treatment IF there has been a clinically important change in retinopathy, OR IF more than 6 weeks have elapsed since the pretreatment photographs.

Submission of pretreatment photographs to the fundus photograph reading center is required only when initiation of panretinal photocoagulation is being considered in an eye that has not previously had such treatment.

If any type of laser photocoagulation (that is, focal/grid or panretinal) is performed, the investigator will report the indication(s) on the appropriate clinical report form page. However, pretreatment photographs should only be obtained for initial panretinal photocoagulation treatments. The fundus photograph reading center will independently evaluate these pre-treatment photographs.

Regardless of when the patient receives laser surgery, the patient will remain on study medication and maintain regularly scheduled study follow-up visits thereafter.

### Efficacy Measures

The following measures will be obtained during the study and will be used to assess efficacy at the specified times:

### Primary:

Best corrected visual acuity will be measured using the ETDRS visual acuity protocol (ETDRS 1985) at Visit 1 and at each visit beginning at Visit 4 and continuing through the patient's last visit.

### Secondary:

ETDRS 7 standard field 30 degree color stereoscopic fundus photography will be performed at Visits 1, 5, 7, 9, 11, 13, 15, and at the patient's last visit to assess the progression of DR and the status of DME.
ETDRS 3 field 30 degree color stereoscopic fundus photography will be performed at Visit 4 to assess the status of DME.

If the sample size is adequate, subgroup analyses may be performed according to compliance status or other baseline characteristics.

### Efficacy Criteria

The primary endpoint of the study is occurrence of SMVL in at least one DR study eye in patients who have moderately severe to very severe NPDR and a best corrected visual acuity of 45 or more letters using the ETDRS visual acuity protocol in at least one eye. If both eyes of a patient have a retinopathy severity level less than 61 without prior panretinal photocoagulation then the patient is defined as having two DR study eyes and the primary endpoint can occur in either eye. If only one eye meets those criteria then the patient has only one DR study eye and the primary endpoint can only occur in that eye. Moderately severe to very severe NPDR (roughly equivalent to the less precisely defined category of preproliferative DR) is defined as Early Treatment Diabetic Retinopathy Study (ETDRS) level of ≧47A and ≦53E as determined using ETDRS 7 standard field 30 degree color stereoscopic fundus photography and the modified Airlie House classification system.

SMVL is defined as the occurrence of ≧15 letters loss in best-corrected ETDRS visual acuity for the 6-month period from Visit 13 (30 months) through Visit 15 (36 months) in at least one DR study eye. Therefore, if visual acuity measurements are available, ≧15 letters loss of visual acuity must occur at Visit 13, Visit 14, and Visit 15 to be considered SMVL. If a patient has ≧15 letters loss at Visit 13 and Visit 15, but Visit 14 has missing visual acuity data, then the patient is still considered to have SMVL. Patients who discontinue to the study early may also have SMVL if there is a 6-month period of ≧15 letters loss in visual acuity ending with the last visit at which visual acuity is assessed (Last Observation Carried Forward, LOCF).

ETDRS Final Retinopathy Severity Scale
Bold type = levels used in change scale Italics = Eligible to be included in this study

| Level | Severity | Definition |
|---|---|---|
| 10 | DR absent | Microaneurysms and other characteristics absent |
| 12a | Non-DR Abnormalities | |
| 14a | DR questionable | 14A HE definite; microaneurysms absent |
| | | 14B SE definite: microaneuryjms absent |
| | | 14C IRMA definite; microaneurysms absent |
| 15a | DR questionable | Hemorrhage(s) definite; microaneurysms absent |
| 20 | Microaneurysms only | Microaneutysms definite; other characteristics absent |
| 35b | Mild NPDR | 35A Venous loops >D/1 |
| | | 35B SE, IRMA, or VB = Q |
| | | 35C Retinal Hemorrhages present |
| | | 35D HE D/1 |
| | | 35E HE M/1 |
| | | 35F SE > D/1 |
| 43 | Moderate NPDR | 43A H/Ma = M/4-5 or S/1 |
| | | 43B IRMA = D/1-3 |
| 47 | [symbol] | 47A Both L43 characteristics |
| | | 47B IRMA = D/4-5 |
| | | 47C H/Mn = S/2-3 |
| | | 47D VB = D/1 |
| 53 | [symbol] | 53A ≧ 2 of the 3 L47 characteristics |
| | | 53B H/Ma ≧ S/4-5 |
| | | 53C IRMA ≧ M/1 |
| | | 53D VB ≧ D/2-3 |
| 53 E | Very Severe NPDR | 53E ≧ 2 or 53B, 53C, and 53D |
| 61 | Mild PDR | 61A FPD and/or FPE only (regressed PDR) |
| | | 61B1 NVE < 1/4 disc area in ≧1 field (Borderline PDR) |
| | | 61B2 NVE ≧ 1/4 but <1/2 disc area in ε1 field |
| 65 | Moderate PDR | 65A NVE > M/1 (≧1/2 disc area in ≧1 field) |
| | | 65B NVD = D and VH or PRH = A or Q |
| | | 65C VH or PRH = D and NVE < M/1 and NVD absent |
| 71, | High-risk PDR | 71A VH or PRH ≧ M/1 (M = about 1 disc area) |
| 75 | | 71B NVE ≧ M/1 and VH or PRH ≧ D/1 |
| | | 71C NVD = D and VH or PRH ≧ D/1 |
| | | 71D NVD ≧ M |
| | | 75 NVD ≧ M and VH or PRH ≧ D/1 |
| 81 | Advanced PDR; Fundus | NVD = cannot grade, or NVD < D and NVE = cannot |
| | partially obscured, center | grade in ≧1 field and absent in all others; and retinal |
| | of macula attached | detachment at center of macula <D |
| 85 | Advanced PDR; Posterior | 85A VH = VS in Field 1 or 2 |
| | fundus obscured, or center | 85B Retinal detachment at center of macula = D |
| | of macula detached | |
| 90 | Cannot grade, even for | |
| | level 81 or 85 | |

aLevels 12, 14, and 15 are not considered separate steps in the scale.
bNPDR levels 35 and above all require presence of microaneurysms.
HE = hard exudates;
SE = soft exudates;
IRMA = intraretinal microvascular abnormalities;
VB = venous beading;
H/Ma = hemorrhages/microaneurysms;
NVE = new vessels elsewhere;
NVD = new vessels on or adjacent to optic disc;
VH = vitreous hemorrhage;
PRH = pre-retinal hemorrhage

Severity categories are of the form (maximum severity/extent), where maximum severity can be absent (A), questionable (Q), definitely present (D), moderate (M), severe (S), or very severe (VS) and extent is the number of photographic fields at that severity level.

### Visual Acuity Grading Scale

Best corrected visual acuity is measured with logarithmic visual acuity charts at a distance of 4 meters, and at 1 meter as well if visual acuity was worse than 20/100. Patients are encouraged to make a maximum effort to read as many lines as possible with each eve.

| Visual Acuity | Number of Letters Correct |
|---|---|
| 20/10 | 99-100 |
| 20/12.5 | 94-98 |
| 20/15 | 89-93 |
| 20/20 | 84-88 |
| 20/25 | 79-83 |
| 20/32 | 74-78 |
| 20/40 | 69-73 |
| 20/50 | 64-68 |
| 20/63 | 59-63 |
| 20/80 | 54-58 |
| 20/100 | 49-53 |
| 20/125 | 44-48 |
| 20/160 | 39-43 |
| 20/200 | 34-38 |
| 20/240 | 29-33 |
| 20/320 | 24-28 |
| 20/400 | 19-23 |
| 20/480 | 14-18 |
| 20/640 | 9-13 |
| 20/800 | 4-8 |
| <5/200 | 0-3 |
| Light Perception | |
| No Light Perception | |

### MCCM (b) Results

Effect of Application of Focal Photocoagulation on Frequency
of Sustained Moderate Visual Loss (SMVL)

% Of Patients with SMVL

| Treatment | No Focal PC | Focal PC |
|---|---|---|
| Placebo | 4.1 | 14.1 |
| RBX 32 mg/d | 2.7 | 8.6 |

1. A method for preserving the vision of a patient suffering from diabetic retinopathy which comprises administering to said patient an effective amount of ruboxistaurin or a pharmaceutical salt thereof wherein said administration is in conjunction with focal or grid laser photocoagulation therapy of said retinopathy.

2. The method according to claim 1 wherein said salt is the mesylate.

3. The method according to claim 2 wherein said salt is the mesylate monohydrate.

4. The method of claims 2 wherein said administration is once per day orally.

5. The method of claim 3 wherein said administration is once per day orally.

6. The method of claim 5 wherein the amount of ruboxistaurin mesylate monohydrate administered is from about 32 mg to about 128 mg/day.

7. The method of claim 6 wherein the amount of ruboxistaurin mesylate monohydrate administered is about 32 mg/day.

8. The method of claim 3 wherein said patient is suffering from moderately severe to very severe non-proliferative diabetic retinopathy.
